(19) United States
(12) Patent Application Publication
(10) Pub. No.: US 2023/0207075 A1
(43) Pub. Date: Jun. 29, 2023

(54) MEDIA FORMULATIONS AND PRODUCTION

(71) Applicant: Nucleus Biologics, San Diego, CA (US)

(72) Inventors: David SHEEHAN, Poway, CA (US); Alyssa MASTER, San Diego, CA (US); Francisco Javier Martinez BECERRA, La Mesa, CA (US)

(73) Assignee: Nucleus Biologics, San Diego, CA (US)

(21) Appl. No.: 17/926,546

(22) PCT Filed: May 19, 2021
(86) PCT No.: PCT/US2021/033249

§ 371 Date: Nov. 18, 2022

Related U.S. Application Data
(60) Provisional application No. 63/027,294, filed on May 19, 2020.

(51) Int. Cl. G16H 10/40; C12M 1/36; C12N 5/00; G06Q 30/0283

(52) U.S. Cl. CPC G16H 10/40; C12M 41/48; C12N 5/0018; G06Q 30/0283

(57) ABSTRACT

Methods, devices, and graphical user interfaces are provided for providing a user interface based on an identified candidate cell culture media formulation, the user interface comprising a plurality of affordances corresponding to different media attributes of the identified candidate cell culture media formulation and receiving, by the computer, a selection of at least one of the plurality of affordances corresponding to different media attributes of the identified candidate cell culture media formulation. In some embodiments, a selected cell culture is identified based on the selection. Further, some embodiments include providing a plurality of ingredients combinable into a plurality of cell culture media formulations; selecting a subset of the plurality of ingredients based on the selected cell culture media formulation; and preparing the selected cell culture media formulation using the subset of the plurality of ingredients.

### RELATED APPLICATION

This application claims priority from U.S. Provisional Application No. 63/027,294, filed May 19, 2020, entitled “CUSTOMIZABLE MEDIA FORMULATIONS AND PRODUCTION,” the contents of which are incorporated by reference in their entirety.

### BACKGROUND

Cell therapy extends and saves lives. Cancer survival rates, for example, have significantly increased as cell therapy solutions advanced. A critical component in cell therapy solutions is the cell culture media used to grow cells. For example, to identify effective cell therapies, cell therapy researchers need to experiment with various cells, reagents, and procedures; precise cell media formulations lead to reproducible research and accelerated therapeutic discoveries. Once a cell is identified, cell therapy companies must grow the cells at large scales to treat many patients; precise cell media formulations support efficient and scalable production. Further, in some cell therapies a patient’s own cells are manipulated in vitro, in cell culture media, and, due to biological and disease treatment differences, have unique nutritional needs. Improved methods for preparing and obtaining cell media formulations are needed.

### BRIEF SUMMARY

Described herein are embodiments that provide improved cell culture media formulation production. Advantageously, some embodiments herein provide a cell therapy researcher the ability to efficiently customize a cell culture media formulation to the needs of their targeted cell(s). Further, some embodiments herein provide prompt feedback to the cell therapy researcher, allowing them to test multiple formulations with small incremental component concentration changes and customize a cell media formulation while ensuring that the customized formulations meets cost and/or formula characteristic requirements. Embodiments herein also reduce production times in cell culture media formulation, contributing to the pace and success of cell therapy advancement.

In one embodiment, a first method for preparing a cell culture media formulation includes: providing, by a computer, a first plurality of affordances corresponding to a plurality of candidate cell culture media formulations; and receiving, by the computer, a selection of at least one of the first plurality of affordances; in response to receiving the selection: (1) identifying a candidate cell culture media formulation; and (2) providing, by the computer, a user interface based on the identified candidate cell culture media formulation, the user interface including a second plurality of affordances corresponding to different media attributes of the identified candidate cell culture media formulation. In some embodiments, the method further includes receiving, by the computer, a selection of at least one of the second plurality of affordances; and in response to receiving the selection: (1) identifying, by the computer, a selected cell culture media formulation, and (2) receiving properties of the selected cell culture media formulation. Further in response to receiving the selection, the method may (1) estimate a production cost of the selected cell culture media formulation based on the received properties and (2) transmit, by the computer, the estimated production cost of the selected cell culture media formulation. Advantageously, such embodiments can provide feedback to cell therapy researchers on the cost and characteristics of their customization, allowing those researchers to further customize in order to meet set criteria. In response to receiving a confirmation, some embodiments of the first method may continue with transmitting, by the computer, the selected cell culture media formulation. The first method may also include: receiving, at a location remote from the computer, the selected cell culture media formulation; providing a plurality of ingredients combinable into a plurality of cell culture media formulations; selecting a subset of the plurality of ingredients based on the selected cell culture media formulation; and preparing the selected cell culture media formulation using the subset of the plurality of ingredients.

In a second method, the first method where in response to receiving the selection, the method further (1) prepares, by the computer, a specification of the selected cell culture media formulation, (2) transmit, by the computer, the specification, and (3) receives, by the computer, a confirmation of the specification. In further embodiments of the second method, in response to receiving a confirmation includes receiving a confirmation of the specification. In some embodiments of the second method, the step of receiving, by the computer, a selection of at least one of the second plurality of affordances and the step of transmitting, by the computer, a specification of the selected cell culture media formulation are separated by 1 week or less. In some embodiments of the second method, the step of receiving, by the computer, a selection of at least one of the second plurality of affordances and the step of transmitting, by the computer, a specification of the selected cell culture media formulation are separated by 1 hour or less. In some embodiments of the second method, the step of receiving, by the computer, a selection of at least one of the second plurality of affordances and the step of transmitting, by the computer, a specification of the selected cell culture media formulation are separated by 15 minutes or less. Advantageously, embodiments of the second method allow a cell therapy researcher to significantly reduce the time necessary to wait and approve a specification, thereby reducing the time between ordering and receiving a media formulation.

In a third method, any of the first and second methods further includes receiving, by the computer, a second selection of at least one of the second plurality of affordances and in response to receiving the selection: (1) estimating a revised production cost and (2) transmitting, by the computer, the revised estimate of the production cost. In some embodiments of the third method, the step of receiving, by the computer, a second selection of at least one of the second plurality of affordances and the step of transmitting, by the computer, the revised estimate of the production cost are separated by 24 hours or less. In some embodiments of the third method, the step of receiving, by the computer, a second selection of at least one of the second plurality of affordances and the step of transmitting, by the computer, the revised estimate of the production cost are separated by 1 hour or less. In some embodiments of the third method, the step of receiving, by the computer, a second selection of at least one of the second plurality of affordances and the step of transmitting, by the computer, the revised estimate of the production cost are separated by 15 minutes or less. Advantageously, embodiments of the third method allow a cell therapy researcher to make changes to a customized formulation and receive updated information on the cost of the changed formulation.

In a fourth method, any of the first through third methods further including: in response to receiving the selection, the method may (1) estimate a performance characteristic of the selected cell culture media formulation based on the received properties, (2) transmit, by the computer, the estimated performance characteristic of the selected cell culture media formulation. In some embodiments of the fourth method, the performance characteristic is an osmolality of the selected cell culture media formulation. In some embodiments of the fourth method, the step of receiving, by the computer, a selection of at least one of the second plurality of affordances and the step of transmitting, by the computer, an estimated performance characteristic of the selected cell culture media formulation are separated by 24 hours or less. In some embodiments of the fourth method, the step of receiving, by the computer, a selection of at least one of the second plurality of affordances and the step of transmitting, by the computer, an estimated performance characteristic of the selected cell culture media formulation are separated by 1 hour or less. In some embodiments of the fourth method, the step of receiving, by the computer, a selection of at least one of the second plurality of affordances and the step of transmitting, by the computer, an estimated performance characteristic of the selected cell culture media formulation are separated by 15 minutes or less. Advantageously, embodiments of the fourth method allow a cell therapy researcher to make changes to a customized formulation and receive updated information on the estimated characteristics of the changed formulation.

In a fifth method, the fourth method where transmitting the estimate of the performance characteristic includes transmitting a revised display of the estimated performance characteristic. In such embodiments, a graphical user interface including the second plurality of affordances is updated when the cell therapy researcher selects one or more of the affordances (e.g., changes a customized media formulation) and thereby advantageously provides a revised graphical user interface for the researcher to receive visual feedback.

In a sixth method, any of the first through fifth methods where preparing a specification of the selected cell culture media formulation includes listing units of measure, storage requirements, shipping requirements, packaging (e.g., bags, bottles, etc.), and the subset of the plurality of ingredients.

In a seventh method, any of the first through fifth methods further including in response to receiving the selection: estimating a production timeline of the selected cell culture media formulation based on the received properties; and transmitting, by the computer, the estimated production timeline. Advantageously, embodiments of the seventh method allow a cell therapy researcher to make changes to a customized formulation and receive updated information on the estimated production timeline of the changed formulation. Such updated information may allow a cell therapy researcher to choose between multiple formulation options with a targeted schedule in mind.

In an eight method, any of the first through seventh methods where receiving properties of the subset of the plurality of ingredients includes receiving (1) cost and/or order fill time rates at the selected grade for a selected quantity and (2) cost and/or order fill time rates at the selected packaging for the selected quantity.

In a ninth method, any of the first through eight methods further including in response to receiving the selection: estimating an environmental impact of the selected cell culture media formulation based on the received properties, and transmitting, by the computer, the estimated environmental impact.

In a tenth method, any of the first through ninth methods where receiving, by the computer, a selection of at least one of the second plurality of affordances includes receiving a selected mass concentration of the selected cell culture media formulation, where estimating a production cost of the selected cell culture media formulation includes receiving a cost of each of the ingredients at the selected mass concentration and estimating a total cost of the selected cell culture media formulation based on the cost of each of the ingredients at the selected mass concentration. In some embodiments of the tenth method, the mass concentration is grams per liter.

In an eleventh method, any of the first through tenth methods where the step of receiving, by the computer, a selection of at least one of the first plurality of affordances and the step of preparing the selected cell culture media formulation using the subset of the plurality of ingredients are separated by 12 weeks or less. In some embodiments of the eleventh method, the step of receiving, by the computer, a selection of at least one of the first plurality of affordances and the step of preparing the selected cell culture media formulation using the subset of the plurality of ingredients are separated by 10 weeks or less. In some embodiments of the eleventh method, the step of receiving, by the computer, a selection of at least one of the first plurality of affordances and the step of preparing the selected cell culture media formulation using the subset of the plurality of ingredients are separated by 8 weeks or less.

In a twelfth method, any of the first through eleventh methods where providing a user interface based on the identified cell culture media formulation includes providing affordances associated with material grade, media format, media testing, glucose content, amino acids, proteins, quantity, and packaging.

In a thirteenth method, any of the first through twelfth methods where preparing the selected media formulation using the subset of the plurality of ingredients includes preparing a liquid media formulation.

In a fourteenth method, any of the first through thirteenth methods where preparing the selected media formulation using the subset of the plurality of ingredients includes preparing a powdered media formulation. Some embodiments of the fourteenth method include packaging the powder media formulation in a pod.

In a fifteenth method, any of the first through fourteenth methods further including: providing, by the computer, a grade-type affordance; receiving, at the computer, a selected grade-type; determining whether the selected grade-type is a GMP grade. In some embodiments, the fifteenth method includes in response to determining that the selected grade-type is a GMP grade: preparing a GMP specification of the selected cell culture media, where the step of preparing a specification of the selected cell culture media includes preparing the GMP specification; and in response to determining that the selected grade-type is not a GMP grade: preparing a non-GMP specification of the selected cell culture media, where the step of preparing a specification of the selected cell culture media includes preparing the non-GMP specification.

In a sixteenth method, any of the first through fifteenth methods further including: providing, by the computer, a plurality of cell-type affordances; receiving, at the computer, a selected cell-type; determining whether the selected cell-type conflicts with the selected cell culture media formulation; and in response to determining that the selected cell-type conflicts with the selected cell culture media formulation: providing a recommendation based on the selected cell culture media formulation.

In a seventeenth method, any of first through sixteenth methods further including: providing, by a computer, a plurality of media-type affordances corresponding to a plurality of candidate media types; receiving, by the computer, a selection of at least one of the plurality of media-type affordances; and in response to receiving the selection of at least one of the plurality of media-type affordances: performing the step of providing, by the computer, the first plurality of affordances corresponding to the plurality of candidate cell culture media formulations.

In an eighteenth method, any of the first through seventeenth methods further including receiving, at the computer, a selection of a packaging configuration. In embodiments of the eighteenth method, a selection of packaging configuration includes a selection of bag material, bag size, number of ports on a bag, and/or material composition of port(s) on the bag.

In some embodiments, a graphical user interface is configured to display any of the displays described in the first through eighteenth methods. In some embodiments, an electronic device includes: a processor; memory; and a program, where the program is stored in the memory and configured to be executed by the processor, the program including instructions for performing any of methods 1-34.

### BRIEF DESCRIPTION OF THE DRAWINGS

FIG. 1 is an exemplary system for producing a cell culture media formulation;

FIG. 2 is an exemplary process of providing a second user interface based on a user’s selection at a first user interface;

FIG. 3 is an exemplary process of providing a second user interface based on a user’s selection of a media attribute affordance at a first user interface;

FIG. 4 is an exemplary process flow for producing a cell culture media formulation.

### DETAILED DESCRIPTION

Cell media formulation is a specialty. Once a therapy company targets a cell for experimentation or production, a precise culture media formulation must be identified to grow the cells and maintain the desired cell attributes, phenotype, or behavior. The formulation is a delicate blend of ingredients. For example, a precise media will supply essential nutrients (e.g., amino acids, carbohydrates, vitamins, minerals), growth factors, proteins, and hormones, and will regulate the physio-chemical environment (e.g., pH buffer, osmotic pressure, temperature). The options for ingredients are expansive and no two cell types have exactly the same needs, making precise formulation a challenge. Without a precise formulation for a cell culture media formulation, cell therapies can and do fail. Precise cell culture media formulations are thus crucial to effective cell therapy.

Known cell culture media formulation processes present barriers to fully realizing the promise of cell therapy. Traditionally, cell therapy researchers and companies choose between two deficient options for obtaining cell media. In the first option, a cell therapy researcher can choose a proprietary formula by its brand name. The advantage of this option is that the price of the brand media is known, but the disadvantage is that the effect of the brand media is not. If the particular cell is new or untested with the brand name media, the cell therapy researcher must trial the brand name media with their cell before knowing the formulation’s full effect. Typically the researcher will add multiple additional reagents to the media to improve the results of the brand name media. It is not uncommon to add six to ten additional ingredients, including amino acids, cytokines, and proteins. This trial and error process can take months to get exactly the desired growth and phenotype characteristics. The major contributor to this lengthy process is typically branded media force scientists to guess at the ingredients and test multiple combinations. Once the final formulation is set, the researcher then has two choices to either make the final media in house or have a media company manufacture custom media for their experimentation, validation, and larger scale production. Given that the time between ordering custom media and receiving the delivery is typically greater than 20 weeks, this forces the cell therapy researcher to wait months before beginning actual experiments or production.

One of the significant delays in that process is the quote cycle. A cell therapy researcher submits a request for a quote, usually in paper form, and does not receive immediate feedback on price or properties of the formulation. Sometime after receiving the request for quote, the cell media formulation producer’s sales team contacts the cell therapy researcher to discuss technical details of the request. The sales team then estimates a cost of the formulation and sends the estimate to the cell therapy researcher. Assuming the cell therapy researcher approves, an internal team (sometimes a cell culture media specialist team and sometimes a different team) then produces a specification of the proposed formulation which is, once again, sent back to the cell therapy researcher for confirmation. If the cell therapy researcher approves, only then can the actual media production begin. This second option has the advantage of producing a formulation that is better tailored to the cell therapy researcher’s particular cell, but the myriad steps add significant time to the formulation production process. This second option takes at least 20 weeks from the request for quote to delivery, and usually much more due to scope changes at different steps. Just like the first option, this second option introduces a significant delay between the cell culture researcher beginning the ordering process and beginning experiments or production. Such delays slows advancement of cell therapy.

Embodiments herein provide for improved cell culture media formulation production including, as one example, reduced production times in cell culture media formulation processes. Advantageously, some embodiments herein provide a cell therapy researcher the ability to efficiently customize a cell culture media formulation to the needs of their targeted cell(s). Further, some embodiments herein provide prompt feedback to the cell therapy researcher, allowing them to assess multiple formulations with small incremental component concentration changes and customize a cell media formulation while ensuring that the customized formulations meets budget, timeline, and/or performance characteristic requirements. More generally, embodiments herein increase the pace and success of cell therapy advancement.

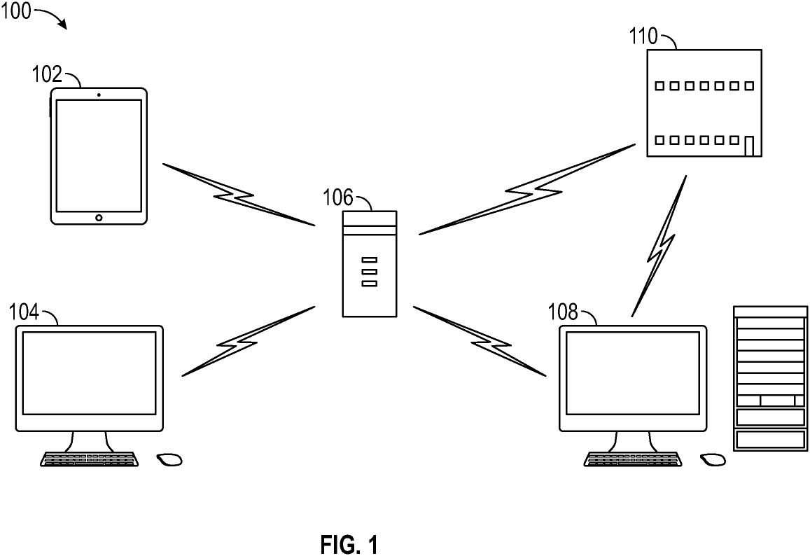

FIG. 1 depicts exemplary system 100 for producing a cell culture media formulation. System 100 includes user devices 102 and 104 capable of processing a user selection of an object displayed in user interfaces on the user devices. As will be appreciated by those skilled in the art, depicted user devices 102 and 104 are exemplary and other portable and stationary devices could be used. Each of user device 102 and 104 could be a tablet, a desktop computer, a smart phone, or any similar device capable of providing graphical objects (e.g., soft keys, icons, web pages, or images) and receiving user input (e.g., touch sensitive display, keyboard, mouse trackpad, etc.). In some embodiments, a user interface includes an affordance (e.g., a user-interactive graphical object) that can provide information on the displayed object’s use and/or can allow the user to provide an input (e.g., a selection). For example, an affordance can include an image (e.g., an icon) that a user modifies to make a selection (e.g., toggling an object’s graphical representation), a search bar for a user to enter text, a hyperlink, a checkbox, an image, etc.

The user interfaces can include affordances that provide a variety of information and/or allow a user to make multiple selections. In some embodiments, the affordances correspond to a plurality of candidate cell culture media formulations. A user selects one of the plurality of candidate cell culture media formulations by interacting with the affordance, where the interaction causes the user interface to change to a second user interface. This change can occur, for example, automatically (for example, the affordance is associated with a hyperlink and when the user selects the hyperlink, the user interface is routed to the linked page), with further action by the user (e.g., the user confirms a selection before proceeding to a new user interface), or with action by a remote server (e.g., the selection is sent to a server that receives the information and transmits a new user interface for display on the user device). Changing a user interface includes any update to a user interface that represents the user’s selection and, for example, includes supplying and displaying an entirely new user interface, and includes supplying and displaying an updated portion of the interacted-with user interface (e.g., by changing the interacted-with affordance).

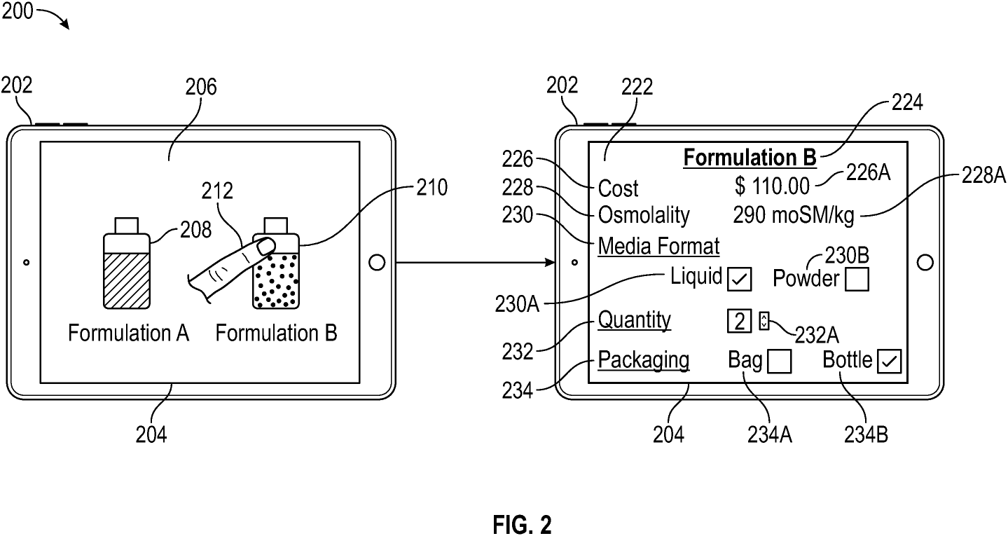

In some embodiments, based on the user’s selection of a candidate cell culture media formulation, system 100 provides a different plurality of affordances corresponding to different media attributes of the identified candidate cell culture media formulation. FIG. 2 depicts exemplary process 200 of providing user interface 222, based on a user’s selection at a first user interface 206, with a second plurality of affordances corresponding to media attributes of the selected formulation.

Process 200 depicts user device 202 with user-interactive display 204. In some embodiments, user device 202 is user device 102 or user device 104 described above with respect to system 100. Process 200 depicts user device 202 with a user interface 206 including affordances 208 and 210 for Formulation A and Formulation B, respectively. Formulations A and B are generic candidate media formulations that a user can select. Other embodiments include one formulation or more than two formulations. In some embodiments, candidate cell culture media formulations include one or more of Dulbecco’s modified eagle’s media, a minimal essential media, a RMPI 1640 media, a medium 199, a Ham’s F-12 media, a McCoy’s media, a Ham’s F-10 media, Eagle’s Minimum Essential medium or Iscove’s modified Dulbecco’s media.

Display 206 depicts a user interaction 212 with affordance 210 corresponding to Formulation B. As will be readily appreciated by those skilled in the art, the illustrated finger is typically not part of the displayed user interface and provided here to aid in the interpretation of the figures. Following the user’s interaction 212 with the interface, a candidate cell culture media formulation is identified. The identification can be processed in the user device or the selection can be transmitted to another device (e.g., server 106 described below) and the identification processed at that device. In response to the identification, a second plurality of affordances corresponding to media attributes of the candidate media formulation are displayed on user interface 222. Media attributes in user interface 222 include a title 224 of the identified candidate media formulation, a cost attribute affordance 226 (and estimated cost affordance 226A), an osmolality attribute affordance 228 (with estimated osmolality affordance 228A), a media format attribute affordance 230 (with affordances for liquid 230A and powder 230B formats), a quantity attribute affordance 232 (with quantity adjustor affordance 232A), and a packaging attribute affordance 234 (with bag option affordance 234A and bottle option affordance 234b).

In some embodiments, other user interfaces (not shown) are displayed before user interface 206, instead of user interface 206, and/or before user interface 222 (which may have follow user interface 206 of another user interaction). In some embodiments, for example, a user interface displays a plurality of media-type affordances, where the media-type affordances correspond to a plurality of candidate media types. For example, some embodiments display media-type affordances for classical, custom, buffers and balanced salt solutions, and cryopreservation media. In response to receiving a selection of at least one of the plurality of media-type affordances, a customizable cell culture media formulation user interface (such as user interface 222) is displayed. In this way, the plurality of media-type affordances can be considered options for a plurality of candidate cell culture media formulations. In other embodiments, selection of a media-type affordance causes a plurality of candidate cell culture media formulation affordances to be displayed on a user interface (e.g., user interface 206) or causes a customizable cell culture media formulation user interface (e.g., user interface 222) to be displayed. In other embodiments, a search bar is displayed on a user interface and a user inserts a search criteria (e.g., a candidate cell culture media formulation, a cell-type, a cell therapy, a manufacturer catalog number, a keyword, etc.) which causes candidate cell culture media formulations to be displayed on a user interface (e.g., user interface 206) or causes a customizable cell culture media formulation user interface (e.g., user interface 222) to be displayed. In other embodiments, cell-type affordances are presented on a user interface and a user selects a cell-type which causes candidate cell culture media formulations to be displayed on a user interface (e.g., user interface 206) or causes a customizable cell culture media formulation user interface (e.g., user interface 222) to be displayed. Exemplary cell-type affordances include affordances for T lymphocytes, B lymphocytes, Natural Killer (NK) cells, dendritic cells, mesenchymal stem cells (MSCs), induced pluripotent stem cells (iPSCs), hematopoietic stem cells (HSCs), islets, and cell lines (e.g. HEK 293 cells).

Returning to user interface 222 of FIG. 2, those skilled in the art will understand that the media attributes depicted there are exemplary and other media attributes are within the scope of this disclosure. For example, some embodiments include a production attribute(s), a performance attribute(s), and/or a component attribute(s). Production attributes include, for example, estimated costs, estimated production timeline (e.g., an estimate of the time for when the end user receives delivery), estimated environmental impact attribute, a media format attribute, a quantity attribute, a packaging attribute, a glucose attribute, a grade attribute, and a testing attribute. Performance attributes include estimated characteristics of the selected formulation, for example, an estimated osmolality and pH. Component attributes include an amino acid attribute, a vitamin attribute, an inorganic salt attribute, a proteins attribute, cytokines and a nucleosides attribute. Other attributes (i.e. other components) such as sugars and antioxidants that do not fall into these categories can be added as well.

Traditionally, the composition of cell culture media formulations is guarded and cell therapy scientists need to experiment (e.g., by adding reagents) to determine whether an obtained cell culture media formulation meets their specific performance criteria. By pre-populating a cell culture media formulation with performance characteristics, embodiments herein allow cell therapy scientists to better predict the effect a cell culture will have on their specific cell. Further, production characteristics can be a gating factor for cell research; by providing the estimated cost with the pre-populated cell culture media formulation user interface, embodiments herein allow cell therapy scientists to determine whether a selected cell culture media formulation is within project specifications. Further embodiments herein allow cell-therapy scientists to customize components of a cell culture media formulation to meet performance and project criteria, as discussed below.

In some embodiments, the cost attribute includes an estimated cost of the media formulation. In some embodiments, the estimated cost is based on cost data stored with the candidate cell culture media formulation’s data. In some embodiments, the estimated cost is determined by accessing the cell culture media formulation’s data and calculating an estimated cost. The cost is estimated by utilizing a database of individual component costs (e.g. individual amino acids, salts, packaging and testing) and adding an estimate of average labor hours required to manufacture the product. In some embodiments, the unit cost of individual component costs are stored at a picogram scale and the media estimate is provided in grams per liter.

In some embodiments, the environmental impact attribute or sustainability index is estimated using information of material sourcing, packaging materials, manufacturing output, waste production and product transportation requirements. In some embodiments, the index changes over time so that end users can track their index over time.

In some embodiments, the quantity attribute allows a user to select the number of units. As shown in user interface 222, the number of units is selected with quantity adjustor 232A, but other embodiments can provide different affordances. For example, a user can manually enter the desired quantity.

In some embodiments, the packaging attribute provides an option between a bottle or a bag, as shown in interface 222. In some embodiments, selection of a packaging option causes display of a further customization for that selection. For example, selecting bottle affordance 234B in user interface 222 may cause display of a bottle size option. In another example, selecting bag affordance 234A in user interface 222 may cause display of affordances for bag material, size, fill volume, and/or ports per bag. In a further example, selecting bag affordance 234A may cause display of affordances for tube material, inside diameter length, outside diameter length, and/or connector fittings. Examples of material choices include platinum cured silicone.

In some embodiments, the media format attribute provides an option between a liquid format and a powder format. As shown in user interface 222, the user can customize the media formulation by selecting a checkbox corresponding to the desired media format. Other embodiments include different affordances, such as a button that can be toggled between liquid and powder.

In some embodiments, a glucose attribute includes options for high glucose, low glucose, no glucose, or other alternatives. In some embodiments, interacting with any of the affordances may provide further information on each (for example, interacting with “high glucose” may generate an information text box with units of glucose per volume of media formulation). In some embodiments, a user selection of an “other” glucose may cause display of a further affordance that provides a manually entered specification.

In some embodiments, a grade attribute includes options for a cGMP or non-cGMP grade. For cGMP materials, grades could include USP-grade raw material while non cGMP made include cell culture grade material.

In some embodiments, a testing attribute includes options for a target pH, osmolality, sterility, mycoplasma, endotoxin, functional, and/or bioburden. Options can be provided for a chosen test method. For example, sterility testing can allow for a choice between the USP <71> method and the rapid sterility method. In another example, mycoplasma testing can have the option of the USP <63> method or the PCR method.

In some embodiments, the performance characteristic attribute (osmolality in user interface 222) includes an estimated performance of the media formulation. In some embodiments, the molecular weight in grams per mole of each of the individual amino acids and other components is identified. Based on the concentration in grams per liter provided by the user, the osmolality of each individual component is calculated. In some embodiments, the total osmolality estimate of the entire formula is calculated by adding these together.

In some embodiments, an amino acid attribute includes options for selecting amino acids to add to/ remove from the media formulation. Exemplary options for amino acids include glycine, hydory L-proline, L-Alanin, L-Alanyl-L-glutamine, L-Arginine, L-Arginine hydrocholoride, L-Aspargine, L-Aspargine-H2O, L-Asparatic Acid, L-Cysteine, L-Cysteine hydrocholoride anydrous, L-Cyseine hydrocholordie-H2O, L-Cystine, L-Cystine-2HCL, L-Cystine-2Na, L-Glutamine acid, L-Glutamine, L-Histidine, L-Histidine-H20, L-Proline, L-Serine, L-Threonine, L-Tryptophan, L-Tyrosine, L-Tyrosine disodium salt dehydrate, and/or L-Valine. In some embodiments, the amino acids can be selected at a cell culture grade or a USP grade, and a particular concentration (e.g., g/L) can be selected. Exemplary amino acids are depicted in the user interfaces of process 300, described further below.

In some embodiments, a vitamins attribute includes options for selecting vitamins to add to/remove from the media formulation. Exemplary options for vitamins may include ascorbic acid, biotin, riboflavin, vitamin B12, niacinamide/nicotinamide, folic acid, thiamine hydrochloride and vitamin A.

In some embodiments, an inorganic salts attribute includes options for selecting salts to add to/remove from the media formulation. Exemplary options for salts include calcium chloride, calcium nitrate, copper sulfate, ferric nitrate, magnesium chloride, potassium chloride, sodium chloride and sodium phosphate dibasic.

In some embodiments, a proteins attribute includes options for selecting AlbuMax II, Human Transferrin (holo), Insulin (recombinant full chain), Human serum Albumin Pharmaceutical grade, Bovine Serum Albumin, and recombinant HSA. In some embodiments, the proteins can be selected at a cell culture grade or a USP grade, and a particular concentration (e.g., g/L) can be selected.

In some embodiments, a nucleosides attribute includes options for selecting nucleosides to add to/remove from the media formulation. Exemplary options for nucleosides include 2′-deoxyadenosine, 2′-deoxyadenosine HCl, adenosine, cytidine, and guanosine.

With reference to system 100, server 106 communicates with user devices 102 and 104. Server 106 can provide graphical objects to the user devices (such as user device 200 displaying in process 200), including user interfaces, affordances, data, etc. Server 106 also receive selections and other inputs from the user devices, process the selections/inputs, and provide updated information for a user interface. For example, server 106 can provide a first plurality of affordances corresponding to a plurality of candidate cell culture media formulations. In some embodiments, server 106 receives a selection of at least one of the first plurality of affordances and in response to receiving the selection: (1) identifies a candidate cell culture media formulation and (2) provides a user interface based on the identified candidate cell culture media formulation, the user interface including a second plurality of affordances corresponding to different media attributes of the identified candidate cell culture media formulation. In some embodiments, server 106 receives a selection of at least one of the second plurality of affordances and in response to receiving the selection: (1) identifies a selected cell culture media formulation and (2) receives properties of the selected cell culture media formulation. In some embodiments, server 106 also, in response to receiving the selection, estimates a production cost of the selected cell culture media formulation based on the received properties and (2) transmits the estimated production cost of the selected cell culture media formulation. In some embodiments, server 106 also, in response to receiving the selection, (1) prepares a specification of the selected cell culture media formulation, (2) transmits the specification, and (3) receives a confirmation of the specification. In some embodiments, server 106 may also transmit the selected cell culture media formulation to a remote location (e.g., production facility 110 described further below).

System 100 also includes administrative computer 108 in communication with server 106. Administrative computer 108 may perform some or all of the functions of server 106. For example, administrative computer 108 may perform the steps of (1) preparing a specification of the selected cell culture media formulation and (2) transmitting the specification. Administrative computer 108 may display user interfaces to allow a cell culture media specialist team to review and/or revise specifications. Such user interfaces can include inputs for ingredients and grade for formulations. Input can include one or more of ingredient type, name, display unit type (e.g., mass or volume), display unit, molecular mass, osmolality, minimum quantity, maximum quantity, reference number, and grade ID.

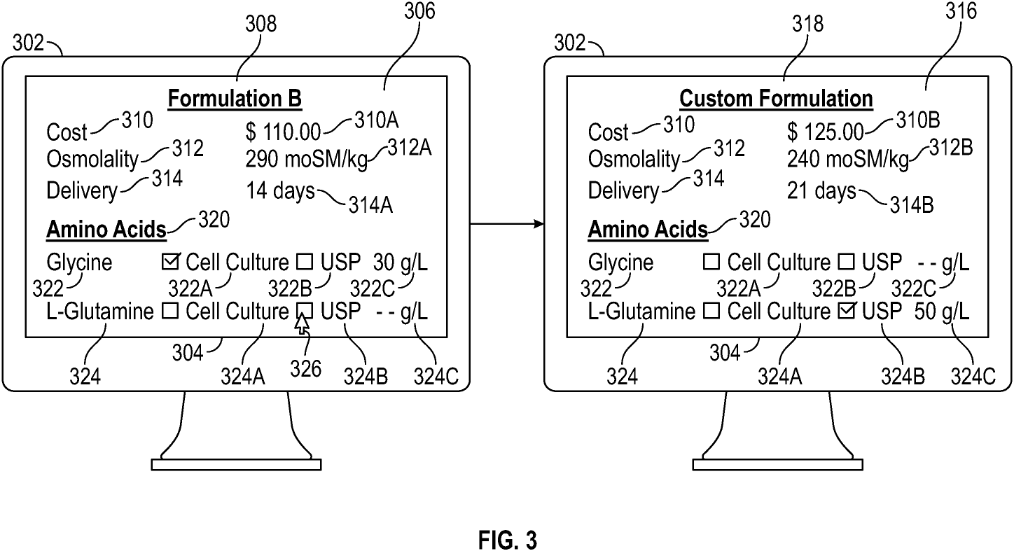

FIG. 3 depicts exemplary process 300 of providing second user interface 316, caused by a user’s selection of a media attribute affordance at a first user interface 306. First user interface 306 may be displayed in response to a user selecting a candidate media formulation, for example, as described with respect to process 200 of FIG. 2. User interfaces 306 and 316 are similar to user interface 222 described above with respect to process 200. User interface 306 and 316 are displayed on display 304 of user device 302. In some embodiments, user device 302 is user device 102 or user device 104 described above with respect to system 100 or user device 202 described above with respect to process 200.

User interfaces 306 and 316 include amino acids attribute affordance 320 with affordances 322 and 324 corresponding to options for amino acids Glycine and L-Glutamine, respectively. Each amino acid affordance has corresponding interactive affordances to allow a user to customize the amino acid characteristics, such as grade (cell culture and U.S. Pharmacopeia) and concentration. In user interface 306, Formulation B is displayed with Glycine as the amino acid at cell culture grade (affordance 322A) and a concentration of “30 g/L” (affordance 322C). As will be appreciated by those skilled in the art, amino acid attribute affordance 320 is described here for illustrative purposes. In some embodiments, other attribute affordances are displayed and customized, including a cost attribute, an environmental impact attribute, a performance characteristic (e.g., osmolality and/or pH) attribute, a production timeline (e.g., delivery time) attribute, a media format attribute, a quantity attribute, a packaging attribute, a glucose attribute, a grade attribute, a testing attribute, a vitamin attribute, an inorganic salt attribute, a proteins attribute, and a nucleosides attribute.

Display 306 depicts a user interaction 326 with media attribute affordance 324A corresponding to a USP Grade for L-Glutamine amino acids. In response to the user’s selection of media attribute affordance 324A, the amino acid attribute of Formulation B has changed to L-Glutamine at U.S. Pharmacopeia grade and concentration of “50 g/L.” In some embodiments, the concentration is updated by the user’s manual entry. In some embodiments, the concentration is automatically updated by an algorithm based on other parameters. In some embodiments, selecting a media attribute affordance causes another attribute affordance to be de-selected (for example, selecting L-Glutamine U.S. Pharmacopoeia grade checkbox 324B caused L-Alanyl L-Glutamine (aka Glutamax) cell culture grade check 322A to be de-selected). In other embodiments, selecting a media attribute affordance may cause other affordances to be un-selectable when, for example, an identified cell-type is inconsistent with certain components and/or an available component is inconsistent with an already selected component. In some embodiments, an end-user can set a limit on cost (or another production attribute and/or performance characteristic) which removes (e.g., causes an affordance to be non-selectable) components, grades, or concentrations that are inconsistent with the cost limit or notifies the user to make these changes.

As a result of the user’s interaction 326, the estimated cost 310B, estimated osmolality 312B, and estimated production timeline 314B are updated, thereby providing feedback to cell therapy researchers on the cost and characteristics of their customized formulation, allowing those researchers to further customize an order to meet their cell’s criteria. As will be readily appreciated by those skilled in the art, the illustrated cursor 326 is provided here to aid in the interpretation of the figures and other visual representations of the user’s interaction could be used.

User interfaces 306 and 316 include titles 308 and 318, respectively. In response to the user’s selection of media attribute affordance 324A, the title of the formulation changes from “Formulation B” to “Custom Formulation,” thereby signifying to the user that the formulation displayed on the user interface is different from a candidate formulation (e.g., the pre-populated media attributes of “Formulation B” on user interface 306 is no longer displayed). In some embodiments, the specific text of title 318 is changed by the user to assist with tracking their customized formulations. In some embodiments, the title of a formulation does not change in response to the user’s selection of a media attribute.

User interfaces 306 and 316 include cost affordance 310 and estimated cost affordances 310A and 310B, respectively. In response to the user’s selection of media attribute affordance 324A, the estimated cost changes from “$110.00” to “$125.00,” thereby signifying to the user that the user’s customization has resulted in an increase in the cost. Some embodiments include an “undo” affordance (not shown), that allows the user to return to a previous media formulation and its estimated cost.

In some embodiments, receiving the customization (e.g., via user interaction 326 with L-Glutamine U.S. Pharmacopeia affordance 324B) and transmitting the revised estimate of the production cost are separated by 24 hours or less. In some embodiments, the separation is 1 hour or less. In some embodiments, receiving the customization and transmitting the revised estimate are separated by 15 minutes or less. Advantageously, embodiments herein allow a cell therapy researcher to make changes to a customized formulation and promptly receive updated information on the cost of the changed formulation. This may reduce cell therapy researchers wait for cell culture media formulations, thereby increasing the pace at which cell therapy advances.

In response to receiving the selection (via user’s interaction 326), a computer can identify a selected cell culture media formulation. Such a computer can include user devices 102 and 104, server 106, and/or administrative computer 108 (discussed further below), and includes any system capable of receiving input of a user’s selection and identifying a selected cell culture media formulation based on the selection, for example. Once the selected cell culture media formulation is identified, the computer receives the properties of the selected cell culture media formulation. Receiving properties may include receiving, for example, one or more of lot size, product configuration (bags or bottles), fill volumes, complexity of formulation and quality testing required.

Based on the received properties, the computer estimates a revised production cost of the selected cell culture media formulation and then transmits the revised estimate of the production cost of the selected cell culture media formulation to the display 304 and user device 302. As used herein, transmission to a display may include transmission from a remote device (e.g., server 106 and/or administrative computer 108) to user device 302 and include transmission from a computer (e.g., a processor) within user device 302 to display 304 of user device 302. The estimated production cost is then displayed as affordance 310B on user interface 316.

In some embodiments, estimating the cost includes receiving cost at the selected grade for a selected quantity and cost fill time rates at the selected packaging for the selected quantity. In some embodiments, receiving properties includes receiving a selected mass concentration of the selected cell culture media formulation and estimating a production cost of the selected cell culture media formulation includes receiving a cost of each of the ingredients at the selected mass concentration and estimating a total cost of the selected cell culture media formulation based on the cost of each of the ingredients at the selected mass concentration. In some embodiments, the mass concentration is grams per liter. In some embodiments the cost based on the total lot size and corresponding fill volumes of the final container.

User interfaces 306 and 316 include osmolality affordance 312 and estimated osmolality affordances 312A and 312B, respectively. In response to the user’s selection of media attribute affordance 324A, the estimated osmolality changes from “290 moSM/kg” to “240 moSM/kg,” thereby signifying to the user that the user’s customization has resulted in a decrease in osmolality. Osmolality affordances 312A and 312B could be replaced by or supplemented with other media formulation performance characteristics. Other exemplary performance characteristics can include pH and/or whether the formula is xeno-free. Some embodiments include an “undo” affordance (not shown) to allow the user to return to a previous media formulation and its estimated osmolality.

In some embodiments, receiving the customization (e.g., via user interaction 326 with L-Glutamine U.S. Pharmacopeia affordance 324B) and transmitting the revised estimate of the performance characteristics of the selected cell culture media formulation are separated by 24 hours or less. In some embodiments, the separation is 1 hour or less. In some embodiments, the steps are separated by 15 minutes or less. In some embodiments, transmitting the estimate of the performance characteristic (e.g., osmolality) includes transmitting a revised display of the estimated performance characteristic. In such embodiments, a graphical user interface including the second plurality of affordances is updated when the cell therapy researcher selects one or more of the affordances (e.g., changes a customized media formulation) and thereby advantageously provides a revised graphical user interface for the cell therapy scientist to receive visual feedback.

In some embodiments, receiving properties may include receiving, for example, one or more of molecular weight and grams per liter. Based on the received properties, the computer estimates a revised performance characteristic of the selected cell culture media formulation and then transmits the revised estimate of the performance characteristic of the selected cell culture media formulation to the display 304 and user device 302. As used herein, transmission to a display may include transmission from a remote device (e.g., server 106 and/or administrative computer 108) to user device 302, include transmission from a computer (e.g., a processor) within user device 302 to display 304 of user device 302, and or any method of communicating the revised estimate to a display. The estimated performance characteristic is then displayed as affordance 312B on user interface 316. The molecular weight in grams per mole of each of the individual amino acids and other components can be identified. Based on the concentration in grams per liter provided by the user, the osmolality of each individual component is calculated. In some embodiments, the total osmolality estimate of the entire formula is calculated by adding these together.

In some embodiments, an end-user can set a limit on a performance characteristic which removes components, grades, or concentrations that are inconsistent with the cost limit. For example, a user interface provides a plurality of cell-type affordances and a method includes: receiving, at a computer, a user’s selected cell-type and determining whether the selected cell-type conflicts with a selected cell culture media formulation. In response to determining that the selected cell-type conflicts with the selected cell culture media formulation, some embodiments provide a recommendation for altering the selected cell culture media formulation consistent with the selected cell-type. In another example, a user interfaces provides affordances for critical quality attributes (for example, desired criteria of cells, such as proliferation, phenotype preservation, or transduction efficiency). The provided recommendation may include explaining the effect (positive or negative) of the customization on the cells. In some embodiments, a selected cell-type conflicts with a selected cell culture media formulation when the cell culture media does not support a desired proliferation, causes loss of viability or apoptosis, and/or other change in the phenotype of the cell. Other examples may be selection of components that are not xeno-free or cGMP certifiable.

User interfaces 306 and 316 include production timeline affordance 314 and estimated production timeline affordances 314A and 314B, respectively. In response to the user’s selection of media attribute affordance 324A, the estimated testing timeline changes from “14 days” to “21 days”. Some embodiments include an “undo” affordance (not shown) to allow the user to return to a previous media formulation and its estimated production timeline.

In some embodiments, receiving properties may include receiving, for example, one or more of availability of ingredients. Based on the received properties, the computer estimates a revised production timeline of the selected cell culture media formulation and then transmits the revised production timeline of the selected cell culture media formulation to the display 304 and user device 302. As used herein, transmission to a display may include transmission from a remote device (e.g., server 106 and/or administrative computer 108) to user device 302 and transmission from a computer (e.g., a processor) within user device 302 to display 304 of user device 302. The estimated production timeline is then displayed as affordance 310B on user interface 316. Advantageously, embodiments herein allow a cell therapy researcher to make changes to a customized formulation and receive updated information on the estimated production timeline of the changed formulation. Such updated information may allow a cell therapy researcher to choose between multiple formulation options with a targeted schedule in mind.

Some embodiments include preparation of a specification of the selected cell culture media formulation and transmission of the specification to a user for confirmation. With reference to FIG. 1, server 106 or administrative computer 108 may determine a specification based on user customization at an end user device (as shown, for example, in process 200 of FIG. 2 and process 300 of FIG. 3). In some embodiments, preparing the specification may begin after receiving an instruction from the user that customization of the selected cell culture media formulation is final. For example, the computer may receive an indication that an end user is satisfied with the estimated cost, performance characteristic, and production timeline of the selected cell culture media formulation). In some embodiments, a cell culture media specialist prepares the specification on a computer, such as administrative computer 108. In some embodiments, the computer prepares the specification autonomously. In other embodiments, the specification includes a list of units of measure, storage requirements, shipping requirements, packaging (e.g., bags, bottles, tubes, etc.), and the subset of the plurality of ingredients. In some embodiments, a user selects a grade-type (GMP/ non-GMP) of a selected cell culture media formulation and preparing the specification includes preparing a specification for that grade type. In some embodiments, the computer allows organizations to create profiles for multiple users, to share specifications across the user base, and control access to proprietary formulations.

Once prepared, the computer transmits the specification to a display of a user device. In some embodiments, transmission is caused by receiving a confirmation from the end user (for example, a confirmation of an estimated cost, an estimated specification, etc.). In some embodiments, receiving the user’s customized cell culture media formulation and transmitting a specification of the customized formulation are separated by 1 week or less. In some embodiments, the separation is 1 hour or less. In some embodiments, receiving the customized formulation and transmission of the specification are separated by 15 minutes or less. Advantageously, embodiments of the second method allow a cell therapy researcher to significantly reduce the time necessary to wait and approve a specification, thereby reducing the time between ordering and receiving a media formulation.

The computer may transmit the selected cell culture media formulation to a remote computer. For example, server 106 or administrative computer 108 may transmit the selected cell culture media formulation to the production facility 110 in system 100, as shown in FIG. 1. Production facility 110 can include a plurality of ingredients capable of combining into a plurality of cell culture media formulations. In some embodiments, the plurality of ingredients are displayed on a customizable cell culture media formulation user interface. For example, the plurality of ingredients can include glucose, amino acids, vitamins, inorganic salts, proteins, and nucleosides, such as those described herein. Based on the selected cell culture media-formulation (e.g., process 300), embodiments herein include selecting a subset of the plurality of ingredients. In some embodiments, selecting a subset of the plurality of ingredients is based on a grade (e.g., cell culture grade, U.S. Pharmacopeia grade) selected at a user interface.

Next, the method includes preparing the selected cell culture media formulation using the subset of the plurality of ingredients. This may include receiving one or more of a selected media format, a selected quantity, a selected packaging, and a selected testing. For example, a chosen media format of liquid or powder is received and an appropriate process is selected to prepare the selected cell culture media formulation. In some embodiments, the process includes providing a powder formulation in a pod. This process includes weighing out the powder components in the formulation, blending those components, adding that powder blend along with any liquid components into a mixing bag and sterile water, solubilizing the powder into the sterile water by mixing, and then dispensing the media through a sterile filter into the final container.

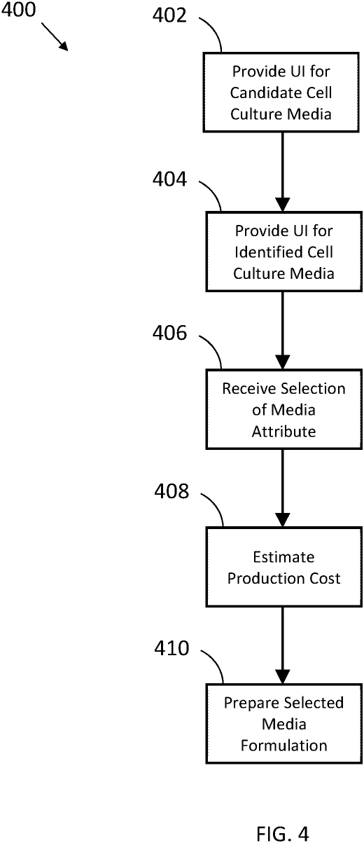

FIG. 4 illustrates process 400 for producing a cell media culture formulation, in accordance with an embodiment. Process 400 includes step 402 of providing, by a computer, a user interface with at least one affordance corresponding to a plurality of candidate cell culture media formulations. In some embodiments, a process 400 includes receiving, by the computer, a selection, via the at least one affordance, of a candidate cell culture media formulation. Process 400 includes, in response to receiving the selection: step 404 of identifying the candidate cell culture media formulation and providing, by the computer, a user interface based on the identified candidate cell culture media formulation. In some embodiments, the user interface includes a plurality of affordances corresponding to different media attributes of the identified candidate cell culture media formulation. Process 400 includes step 406: receiving, by the computer, a selection of at least one of the plurality of affordances corresponding to different media attributes of the identified candidate cell culture media formulation. Step 408 includes, in response to receiving the selection, estimating a production cost of the selected cell culture media formulation based on received properties. Process 400 may also include identifying, by the computer, a selected cell culture media formulation, and receiving properties of the selected cell culture media formulation. Once the production cost is estimated, process 400 can include transmitting, by the computer, the estimated production cost of the selected cell culture media formulation. Process 400 also includes step 410 of, in response to receiving a confirmation, preparing the selected cell culture media formulation using a subset of a plurality of ingredients, the plurality combinable into a plurality of cell culture media formulations. In some embodiments, preparing the selected cell culture includes: transmitting, by the computer, the selected cell culture media formulation; receiving, at a location remote from the computer, the selected cell culture media formulation; providing a plurality of ingredients combinable into a plurality of cell culture media formulations; and selecting the subset of the plurality of ingredients based on the selected cell culture media formulation.

In some embodiments of process 400, providing a user interface with at least one affordance corresponding to a plurality of candidate cell culture media formulations includes providing a plurality of affordances corresponding to the plurality of candidate cell culture media formulations. In some embodiments of process 400, where providing a user interface with at least one affordance corresponding to a plurality of candidate cell culture media formulations includes providing a search bar.

In some embodiments of process 400, in response to receiving the selection, the process further includes: preparing, by the computer, a specification of the selected cell culture media formulation, and transmitting, by the computer, the specification, and where the confirmation include a confirmation of the specification. In some embodiments, the step of receiving, by the computer, a selection of at least one of the plurality of affordances and the step of transmitting, by the computer, a specification of the selected cell culture media formulation are separated by 1 week or less. In some embodiments of process 400, the step of receiving, by the computer, a selection of at least one of the plurality of affordances and the step of transmitting, by the computer, a specification of the selected cell culture media formulation are separated by 1 hour or less. In some embodiments of process 400, the step of receiving, by the computer, a selection of at least one of the plurality of affordances and the step of transmitting, by the computer, a specification of the selected cell culture media formulation are separated by 15 minutes or less. In some embodiments of process 400, preparing a specification of the selected cell culture media formulation includes listing units of measure, storage requirements, shipping requirements, packaging, and the subset of the plurality of ingredients.

In some embodiments of process 400, the process further includes: receiving, by the computer, a second selection of at least one of the plurality of affordances corresponding to different media attributes of the identified candidate cell culture media formulation; and in response to receiving the selection: estimating a revised production cost; and transmitting, by the computer, the revised estimate of the production cost. In some embodiments of process 400, the step of receiving, by the computer, a second selection of at least one of the plurality of affordances and the step of transmitting, by the computer, the revised estimate of the production cost are separated by 24 hours. In some embodiments of process 400, the step of receiving, by the computer, a second selection of at least one of the plurality of affordances and the step of transmitting, by the computer, the revised estimate of the production cost are separated by 1 hour. In some embodiments of process 400, the step of receiving, by the computer, a second selection of at least one of the plurality of affordances and the step of transmitting, by the computer, the revised estimate of the production cost are separated by 15 minutes.

In some embodiments of process 400, the process further includes: in response to receiving the selection of at least one of the plurality of affordances: estimating a performance characteristic of the selected cell culture media formulation; and transmitting, by the computer, the revised estimate of the performance characteristic. In some embodiments of process 400, the step of receiving, by the computer, a selection of at least one of the plurality of affordances and the step of transmitting, by the computer, the estimate of the performance characteristic are separated by 24 hours. In some embodiments of process 400, the step of receiving, by the computer, a selection of at least one of the plurality of affordances and the step of transmitting, by the computer, the estimate of the performance characteristic are separated by 1 hour. In some embodiments of process 400, the step of receiving, by the computer, a selection of at least one of the plurality of affordances and the step of transmitting, by the computer, the estimate of the performance characteristic are separated by 15 minutes. In some embodiments of process 400, the performance characteristic includes at least one of osmolality and pH.

In some embodiments of process 400, the process further includes in response to receiving the selection: estimating a production timeline of the selected cell culture media formulation based on the received properties; and transmitting, by the computer, the estimated production timeline.

In some embodiments of process 400, receiving properties of the subset of the plurality of ingredients includes receiving (1) cost and fill time rates at the selected grade for a selected quantity and (2) cost and fill time rates at the selected packaging for the selected quantity.

In some embodiments of process 400, the process further includes in response to receiving the selection: estimating an environmental impact of the selected cell culture media formulation based on the received properties, and transmitting, by the computer, the estimated environmental impact.

In some embodiments of process 400, receiving, by the computer, a selection of at least one of the plurality of affordances includes receiving a selected mass concentration of the selected cell culture media formulation, where estimating a production cost of the selected cell culture media formulation includes receiving a cost of each of the ingredients at the selected mass concentration and estimating a total cost of the selected cell culture media formulation based on the cost of each of the ingredients at the selected mass concentration. In some embodiments of process 400, the mass concentration is grams per liter.

In some embodiments of process 400, the step of receiving, by the computer, a selection of the candidate cell culture media formulation and the step of preparing the selected cell culture media formulation using the subset of the plurality of ingredients are separated by 12 weeks or less. In some embodiments of process 400, the step of receiving, by the computer, a selection of the candidate cell culture media formulation and the step of preparing the selected cell culture media formulation using the subset of the plurality of ingredients are separated by 10 weeks or less. In some embodiments of process 400, the step of receiving, by the computer, a selection of the candidate cell culture media formulation and the step of preparing the selected cell culture media formulation using the subset of the plurality of ingredients are separated by 8 weeks or less.

In some embodiments of process 400, providing a user interface based on the identified cell culture media formulation includes providing affordances associated with material grade, media format, media testing, glucose content, amino acids, serum, quantity, osmolality; and packaging.

In some embodiments of process 400, preparing the selected media formulation using the subset of the plurality of ingredients includes preparing a liquid media formulation.

In some embodiments of process 400, preparing the selected media formulation using the subset of the plurality of ingredients includes preparing a powdered media formulation. In some embodiments of process 400, preparing a powder media formulation includes packaging the powder media formulation in a pod.

In some embodiments of process 400, the process further includes: providing, by the computer, a grade-type affordance; receiving, at the computer, a selected grade-type; determining whether the selected grade-type is an FDA grade; in response to determining that the selected grade-type is an FDA grade: preparing a GMP specification of the selected cell culture media, where the step of preparing a specification of the selected cell culture media includes preparing the GMP specification; and in response to determining that the selected grade-type is not an FDA grade: preparing a non-GMP specification of the selected cell culture media, where the step of preparing a specification of the selected cell culture media includes preparing the non-GMP specification.

In some embodiments of process 400, the process further includes providing, by the computer, a plurality of cell-use affordances; receiving, at the computer, a selected cell-type; determining whether the selected cell-type conflicts with the selected cell culture media formulation; and in response to determining that the selected cell-type conflicts with the selected cell culture media formulation: providing a recommendation based on the selected cell culture media formulation.

In some embodiments of process 400, the process further includes providing, by a computer, a plurality of media-type affordances corresponding to a plurality of candidate media types; receiving, by the computer, a selection of at least one of the plurality of media-type affordances; and in response to receiving the selection of at least one of the plurality of media-type affordances: performing the step of providing, by the computer, the user interface with at least one affordance corresponding to a plurality of candidate cell culture media formulations.

In some embodiments of process 400, further includes receiving, at the computer, a selection of a packaging configuration. In some embodiments of process 400, selection of a packaging configuration includes a selection of bag material, bag size, number of ports on a bag, and/or material composition of port(s) on the bag.

In some embodiments, a graphical user interface configured to display any of the displays described in various embodiments of process 400.

In some embodiments, an electronic device includes: a processor; memory; and a program, where the program is stored in the memory and configured to be executed by the processor, the program including instructions for performing any of the various embodiments of process 400.

A series of exemplary embodiments will now be described.

1. A method for preparing a cell culture media formulation, comprising: providing, by a computer, a user interface with at least one affordance corresponding to a plurality of candidate cell culture media formulations; receiving, by the computer, a selection, via the at least one affordance, of a candidate cell culture media formulation; in response to receiving the selection: identifying the candidate cell culture media formulation; providing, by the computer, a user interface based on the identified candidate cell culture media formulation, the user interface comprising a plurality of affordances corresponding to different media attributes of the identified candidate cell culture media formulation; receiving, by the computer, a selection of at least one of the plurality of affordances corresponding to different media attributes of the identified candidate cell culture media formulation; in response to receiving the selection: identifying, by the computer, a selected cell culture media formulation, receiving properties of the selected cell culture media formulation, estimating a production cost of the selected cell culture media formulation based on the received properties, and transmitting, by the computer, the estimated production cost of the selected cell culture media formulation; in response to receiving a confirmation: transmitting, by the computer, the selected cell culture media formulation; receiving, at a location remote from the computer, the selected cell culture media formulation; providing a plurality of ingredients combinable into a plurality of cell culture media formulations; selecting a subset of the plurality of ingredients based on the selected cell culture media formulation; and preparing the selected cell culture media formulation using the subset of the plurality of ingredients.

2. The method of exemplary embodiment 1, wherein providing a user interface with at least one affordance corresponding to a plurality of candidate cell culture media formulations comprises providing a plurality of affordances corresponding to the plurality of candidate cell culture media formulations.

3. The method of exemplary embodiments 1 or 2, wherein providing a user interface with at least one affordance corresponding to a plurality of candidate cell culture media formulations comprises providing a search bar.

4. The method of any of exemplary embodiments 1-3, wherein in response to receiving the selection, the method further comprising: preparing, by the computer, a specification of the selected cell culture media formulation, and transmitting, by the computer, the specification, and wherein the confirmation comprises a confirmation of the specification.

5. The method of exemplary embodiment 4, wherein the step of receiving, by the computer, a selection of at least one of the plurality of affordances and the step of transmitting, by the computer, a specification of the selected cell culture media formulation are separated by 1 week or less.

6. The method of exemplary embodiment 5, wherein the step of receiving, by the computer, a selection of at least one of the plurality of affordances and the step of transmitting, by the computer, a specification of the selected cell culture media formulation are separated by 1 hour or less.

7. The method of exemplary embodiment 6, wherein the step of receiving, by the computer, a selection of at least one of the plurality of affordances and the step of transmitting, by the computer, a specification of the selected cell culture media formulation are separated by 15 minutes or less.

8. The method of any of exemplary embodiments 4-7, wherein preparing a specification of the selected cell culture media formulation comprises listing units of measure, storage requirements, shipping requirements, packaging, and the subset of the plurality of ingredients.

9. The method of any of exemplary embodiments 1-8, further comprising: receiving, by the computer, a second selection of at least one of the plurality of affordances corresponding to different media attributes of the identified candidate cell culture media formulation; and in response to receiving the selection: estimating a revised production cost; and transmitting, by the computer, the revised estimate of the production cost.

10. The method of exemplary embodiment 9, wherein the step of receiving, by the computer, a second selection of at least one of the plurality of affordances and the step of transmitting, by the computer, the revised estimate of the production cost are separated by 24 hours.

11. The method of exemplary embodiment 10, wherein the step of receiving, by the computer, a second selection of at least one of the plurality of affordances and the step of transmitting, by the computer, the revised estimate of the production cost are separated by 1 hour.

12. The method of exemplary embodiment 11, wherein the step of receiving, by the computer, a second selection of at least one of the plurality of affordances and the step of transmitting, by the computer, the revised estimate of the production cost are separated by 15 minutes.

13. The method of any of exemplary embodiments 1-12, further comprising: in response to receiving the selection of at least one of the plurality of affordances: estimating a performance characteristic of the selected cell culture media formulation; and transmitting, by the computer, the revised estimate of the performance characteristic.

14. The method of exemplary embodiment 13, wherein the step of receiving, by the computer, a selection of at least one of the plurality of affordances and the step of transmitting, by the computer, the estimate of the performance characteristic are separated by 24 hours.

15. The method of exemplary embodiment 14, wherein the step of receiving, by the computer, a selection of at least one of the plurality of affordances and the step of transmitting, by the computer, the estimate of the performance characteristic are separated by 1 hour.

16. The method of exemplary embodiment 15, wherein the step of receiving, by the computer, a selection of at least one of the plurality of affordances and the step of transmitting, by the computer, the estimate of the performance characteristic are separated by 15 minutes.

17. The method of any of exemplary embodiments 13-16, wherein the performance characteristic includes at least one of osmolality and pH.

18. The method of any of exemplary embodiments 1-17, further comprising: in response to receiving the selection: estimating a production timeline of the selected cell culture media formulation based on the received properties; and transmitting, by the computer, the estimated production timeline.

19. The method of any of exemplary embodiments 1-18, wherein receiving properties of the subset of the plurality of ingredients comprises receiving (1) cost and fill time rates at the selected grade for a selected quantity and (2) cost and fill time rates at the selected packaging for the selected quantity.

20. The method of any of exemplary embodiments 1-19, further comprising: in response to receiving the selection: estimating an environmental impact of the selected cell culture media formulation based on the received properties, and transmitting, by the computer, the estimated environmental impact.

21. The method of any of exemplary embodiments 1-20, wherein receiving, by the computer, a selection of at least one of the plurality of affordances comprises receiving a selected mass concentration of the selected cell culture media formulation, wherein estimating a production cost of the selected cell culture media formulation comprises receiving a cost of each of the ingredients at the selected mass concentration and estimating a total cost of the selected cell culture media formulation based on the cost of each of the ingredients at the selected mass concentration.

22. The method of exemplary embodiment 21, wherein the mass concentration is grams per liter.

23. The method of any of exemplary embodiments 1-22, wherein the step of receiving, by the computer, a selection of the candidate cell culture media formulation and the step of preparing the selected cell culture media formulation using the subset of the plurality of ingredients are separated by 12 weeks or less.

24. The method of exemplary embodiment 23, wherein the step of receiving, by the computer, a selection of the candidate cell culture media formulation and the step of preparing the selected cell culture media formulation using the subset of the plurality of ingredients are separated by 10 weeks or less.

25. The method of exemplary embodiment 24, wherein the step of receiving, by the computer, a selection of the candidate cell culture media formulation and the step of preparing the selected cell culture media formulation using the subset of the plurality of ingredients are separated by 8 weeks or less.

26. The method of any of exemplary embodiments 1-25, wherein providing a user interface based on the identified cell culture media formulation comprises providing affordances associated with material grade, media format, media testing, glucose content, amino acids, serum, quantity, osmolality; and packaging.

27. The method of any of exemplary embodiments 1-26, wherein preparing the selected media formulation using the subset of the plurality of ingredients comprises preparing a liquid media formulation.

28. The method of any of exemplary embodiments 1-27, wherein preparing the selected media formulation using the subset of the plurality of ingredients comprises preparing a powdered media formulation.

29. The method of exemplary embodiment 28, wherein preparing a powder media formulation comprises packaging the powder media formulation in a pod.

30. The method of any of exemplary embodiments 1-29, further comprising: providing, by the computer, a grade-type affordance; receiving, at the computer, a selected grade-type; determining whether the selected grade-type is an FDA grade; in response to determining that the selected grade-type is an FDA grade: preparing a GMP specification of the selected cell culture media, wherein the step of preparing a specification of the selected cell culture media comprises preparing the GMP specification; and in response to determining that the selected grade-type is not an FDA grade: preparing a non-GMP specification of the selected cell culture media, wherein the step of preparing a specification of the selected cell culture media comprises preparing the non-GMP specification.

31. The method of any of exemplary embodiments 1-30, further comprising: providing, by the computer, a plurality of cell-use affordances; receiving, at the computer, a selected cell-type; determining whether the selected cell-type conflicts with the selected cell culture media formulation; and in response to determining that the selected cell-type conflicts with the selected cell culture media formulation: providing a recommendation based on the selected cell culture media formulation.

32. The method of any of exemplary embodiments 1-31, further comprising: providing, by a computer, a plurality of media-type affordances corresponding to a plurality of candidate media types; receiving, by the computer, a selection of at least one of the plurality of media-type affordances; and in response to receiving the selection of at least one of the plurality of media-type affordances: performing the step of providing, by the computer, the user interface with at least one affordance corresponding to a plurality of candidate cell culture media formulations.

33. The method of any of exemplary embodiments 1-32, further comprising receiving, at the computer, a selection of a packaging configuration.

34. The method of exemplary embodiment 33, wherein selection of a packaging configuration includes a selection of bag material, bag size, number of ports on a bag, and/or material composition of port(s) on the bag.

35. A graphical user interface configured to display any of the displays described in embodiments 1-34.

36. An electronic device, comprising: a processor; memory; and a program, wherein the program is stored in the memory and configured to be executed by the processor, the program comprising instructions for performing any of embodiments 1-34.

Numerous modifications may be made to the foregoing systems without departing from the basic teachings thereof. Although the devices and methods have been described in substantial detail with reference to one or more specific embodiments, those of skill in the art will recognize that changes may be made to the embodiments specifically disclosed in this application, yet these modifications and improvements are within the scope and spirit of the disclosure. The terminology used in the description of the various described embodiments herein is for the purpose of describing particular embodiments only and is not intended to be limiting. As used in the description of the various described embodiments and the appended claims, the singular forms “a”, “an,” and “the” are intended to include the plural forms as well, unless the context clearly indicates otherwise. Similarly, the terms “one or more” and “at least one” include both singular and plural forms, unless the context clearly indicates otherwise. Further, use of the terms “at least one,” “one or more,” and similar terms should not be construed to limit “a”, “an,” and “the” to singular forms. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Although the following description uses terms “first,” “second,” etc. to describe various elements, these elements should not be limited by the terms. These terms are only used to distinguish one element from another. For example, a first user interface could be termed a second user interface, and, similarly, a second user interface could be termed a first user interface, without departing from the scope of the various described embodiments. The first user interface and the second user interface are both user interfaces, but they are not the same user interface.

1. A method for preparing a cell culture media formulation, comprising:
providing, by a computer, a user interface with at least one affordance corresponding to a plurality of candidate cell culture media formulations;
receiving, by the computer, a selection, via the at least one affordance, of a candidate cell culture media formulation;
in response to receiving the selection:
identifying the candidate cell culture media formulation;
providing, by the computer, a user interface based on the identified candidate cell culture media formulation, the user interface comprising a plurality of affordances corresponding to different media attributes of the identified candidate cell culture media formulation;
receiving, by the computer, a selection of at least one of the plurality of affordances corresponding to different media attributes of the identified candidate cell culture media formulation;
in response to receiving the selection:
identifying, by the computer, a selected cell culture media formulation,
receiving properties of the selected cell culture media formulation,
estimating a production cost of the selected cell culture media formulation based on the received properties, and
transmitting, by the computer, the estimated production cost of the selected cell culture media formulation;
in response to receiving a confirmation:
transmitting, by the computer, the selected cell culture media formulation;
receiving, at a location remote from the computer, the selected cell culture media formulation;
providing a plurality of ingredients combinable into a plurality of cell culture media formulations;
selecting a subset of the plurality of ingredients based on the selected cell culture media formulation; and
preparing the selected cell culture media formulation using the subset of the plurality of ingredients.

2. The method of claim 1, wherein providing a user interface with at least one affordance corresponding to a plurality of candidate cell culture media formulations comprises providing a plurality of affordances corresponding to the plurality of candidate cell culture media formulations.

3. The method of claim 1, wherein providing a user interface with at least one affordance corresponding to a plurality of candidate cell culture media formulations comprises providing a search bar.

4. The method of claim 1, wherein
in response to receiving the selection, the method further comprising:
preparing, by the computer, a specification of the selected cell culture media formulation, and
transmitting, by the computer, the specification, and wherein
the confirmation comprises a confirmation of the specification.

5-8. (canceled)

9. The method of claim 1, further comprising:
receiving, by the computer, a second selection of at least one of the plurality of affordances corresponding to different media attributes of the identified candidate cell culture media formulation; and
in response to receiving the selection:
estimating a revised production cost; and
transmitting, by the computer, the revised estimate of the production cost.

10-12. (canceled)

13. The method of claim 1, further comprising:
in response to receiving the selection of at least one of the plurality of affordances:
estimating a performance characteristic of the selected cell culture media formulation; and
transmitting, by the computer, the revised estimate of the performance characteristic.

14-17. (canceled)

18. The method of claim 1, further comprising:
in response to receiving the selection:
estimating a production timeline of the selected cell culture media formulation based on the received properties; and
transmitting, by the computer, the estimated production timeline.

19. The method of claim 1, wherein receiving properties of the subset of the plurality of ingredients comprises receiving (1) cost and fill time rates at the selected grade for a selected quantity and (2) cost and fill time rates at the selected packaging for the selected quantity.

20. The method of claim 1, further comprising:
in response to receiving the selection:
estimating an environmental impact of the selected cell culture media formulation based on the received properties, and
transmitting, by the computer, the estimated environmental impact.

21. The method of claim 1, wherein receiving, by the computer, a selection of at least one of the plurality of affordances comprises receiving a selected mass concentration of the selected cell culture media formulation, wherein estimating a production cost of the selected cell culture media formulation comprises receiving a cost of each of the ingredients at the selected mass concentration and estimating a total cost of the selected cell culture media formulation based on the cost of each of the ingredients at the selected mass concentration.

22. (canceled)

23. The method of claim 1, wherein the step of receiving, by the computer, a selection of the candidate cell culture media formulation and the step of preparing the selected cell culture media formulation using the subset of the plurality of ingredients are separated by 12 weeks or less.

24-25. (canceled)

26. The method of claim 1, wherein providing a user interface based on the identified cell culture media formulation comprises providing affordances associated with material grade, media format, media testing, glucose content, amino acids, serum, quantity, osmolality; and packaging.

27. The method of claim 1, wherein preparing the selected media formulation using the subset of the plurality of ingredients comprises preparing a liquid media formulation.

28. The method of claim 1, wherein preparing the selected media formulation using the subset of the plurality of ingredients comprises preparing a powdered media formulation.

29. (canceled)

30. The method of claim 1, further comprising:
providing, by the computer, a grade-type affordance;
receiving, at the computer, a selected grade-type;
determining whether the selected grade-type is an FDA grade;
in response to determining that the selected grade-type is an FDA grade:
preparing a GMP specification of the selected cell culture media, wherein the step of preparing a specification of the selected cell culture media comprises preparing the GMP specification; and
in response to determining that the selected grade-type is not an FDA grade:
preparing a non-GMP specification of the selected cell culture media, wherein the step of preparing a specification of the selected cell culture media comprises preparing the non-GMP specification.

31. The method of claim 1, further comprising:
providing, by the computer, a plurality of cell-use affordances;
receiving, at the computer, a selected cell-type;
determining whether the selected cell-type conflicts with the selected cell culture media formulation; and
in response to determining that the selected cell-type conflicts with the selected cell culture media formulation:
providing a recommendation based on the selected cell culture media formulation.

32. The method of claim 1, further comprising:
providing, by a computer, a plurality of media-type affordances corresponding to a plurality of candidate media types;
receiving, by the computer, a selection of at least one of the plurality of media-type affordances; and
in response to receiving the selection of at least one of the plurality of media-type affordances:
performing the step of providing, by the computer, the user interface with at least one affordance corresponding to a plurality of candidate cell culture media formulations.

33. The method of claim 1, further comprising receiving, at the computer, a selection of a packaging configuration.

34-35. (canceled)

36. An electronic device, comprising:
a display for displaying user interfaces,
a processor;
memory; and
a program, wherein the program is stored in the memory and configured to be executed by the processor, the program comprising instructions for performing a method comprising:
providing a user interface with at least one affordance corresponding to a plurality of candidate cell culture media formulations;
receiving, a selection, via the at least one affordance, of a candidate cell culture media formulation;
in response to receiving the selection:
identifying the candidate cell culture media formulation;
providing a user interface based on the identified candidate cell culture media formulation, the user interface comprising a plurality of affordances corresponding to different media attributes of the identified candidate cell culture media formulation;
receiving a selection of at least one of the plurality of affordances corresponding to different media attributes of the identified candidate cell culture media formulation;
in response to receiving the selection:
identifying a selected cell culture media formulation,
receiving properties of the selected cell culture media formulation,
estimating a production cost of the selected cell culture media formulation
based on the received properties, and
transmitting the estimated production cost of the selected cell culture media formulation;
in response to receiving a confirmation:
transmitting the selected cell culture media formulation;
receiving, at a location remote from the computer, the selected cell culture media formulation;
providing a plurality of ingredients combinable into a plurality of cell culture media formulations;
selecting a subset of the plurality of ingredients based on the selected cell culture media formulation; and
preparing the selected cell culture media formulation using the subset of the plurality of ingredients.

37. A non-transitory computer-readable medium storing instructions that, when executed by one or more processors, cause the one or more processors to execute a method comprising:
providing a user interface with at least one affordance corresponding to a plurality of candidate cell culture media formulations;
receiving, a selection, via the at least one affordance, of a candidate cell culture media formulation;
in response to receiving the selection:
identifying the candidate cell culture media formulation;
providing a user interface based on the identified candidate cell culture media formulation, the user interface comprising a plurality of affordances corresponding to different media attributes of the identified candidate cell culture media formulation;
receiving a selection of at least one of the plurality of affordances corresponding to different media attributes of the identified candidate cell culture media formulation;
in response to receiving the selection:
identifying a selected cell culture media formulation,
receiving properties of the selected cell culture media formulation,
estimating a production cost of the selected cell culture media formulation based on the received properties, and
transmitting the estimated production cost of the selected cell culture media formulation;
in response to receiving a confirmation:
transmitting the selected cell culture media formulation;
receiving, at a location remote from the computer, the selected cell culture media formulation;
providing a plurality of ingredients combinable into a plurality of cell culture media formulations;
selecting a subset of the plurality of ingredients based on the selected cell culture media formulation; and
preparing the selected cell culture media formulation using the subset of the plurality of ingredients.
